(19) United States
(12) Patent Application Publication
(10) Pub. No.: US 2023/0386613 A1
(43) Pub. Date: Nov. 30, 2023

(54) METHOD FOR EVALUATING PERFORMANCE OF LUBRICATING LAYER

(71) Applicant: SHOWA DENKO, Tokyo (JP)

(72) Inventors: Kohei OCHIAI, Tokyo (JP); Shohei NISHIZAWA, Tokyo (JP); Yoshishige OKUNO, Tokyo (JP); Tsuyoshi KATO, Tokyo (JP)

(21) Appl. No.: 18/248,591

(22) PCT Filed: Oct. 13, 2021
(86) PCT No.: PCT/JP2021/037849

§ 371 Date: Apr. 11, 2023

(30) Foreign Application Priority Data
Oct. 16, 2020 (JP) 2020-174602

(51) Int. Cl. G16C 10/00

(52) U.S. Cl. CPC G16C 10/00

(57) ABSTRACT

A method for evaluating a performance of a lubricating layer according to the present invention includes step of preparing a lubricant molecular model and a protective layer model, using quantum chemical calculation, step of performing molecular dynamics calculation of a lubricating layer initial model constructed from the lubricant molecular model and the protective layer model, and step of calculating an index of a degree of adsorption of lubricant molecules to the protective layer from a result of the molecular dynamics calculation.

### TECHNICAL FIELD

The present invention relates to methods for evaluating performance of lubricating layers.

### BACKGROUND ART

There are demands to reduce a flying height of a magnetic head, in order to improve the recording density of a magnetic recording and reproducing apparatus, such as a hard disk drive or the like. For this reason, there are demands to further reduce the thickness of a lubricating layer that is formed as the outermost surface layer on a protective layer of the magnetic recording medium. At present, the lubricating layer has reached a thickness of 10 Å or less, that is of a monomolecular film level.

A performance of the lubricating layer of the magnetic recording medium, having such a thickness of the monomolecular film level, depends on the various microstructures of lubricant molecules forming the lubricating layer. Hence, lubricant molecules have been proposed by performing a microscale simulation focusing on the microstructure of the lubricant molecules, utilizing a molecular dynamics calculation or a quantum mechanics calculation (for example, refer to Patent Documents 1 and 2).

### PRIOR ART DOCUMENTS

### Patent Documents

Patent Document 1: Japanese Laid-Open Patent Publication No. 2010-168512

Patent Document 2: International Publication Pamphlet No. WO 2018/159232

### DISCLOSURE OF THE INVENTION

### Problems to be Solved by the Invention

The performance of the lubricating layer is defined by macroscopic phenomena typified by lubrication and flying. Such phenomena are observed in experiments, and the performance, such as a lubricating performance, a flying performance, or the like, is evaluated from the observation results. For this reason, in a simulation covering the lubricating layer, there are demands to predict and quantify such macroscopic phenomena.

For example, Patent Document 1 performs a simulation of a state where one lubricant molecule is adsorbed on a surface of the protective film, and a reduction of a surface energy of the lubricating layer is suggested from an orientation of a polar group of the lubricant molecule.

On the other hand, Patent Document 2 performs a simulation by assuming a case where a plurality of molecules are covered under a periodic boundary condition, and the lubricating layer is formed on the outermost surface of the magnetic recording medium. From this simulation, a film thickness and a coverage of the lubricating layer are simulated.

However, neither Patent Document 1 nor Patent Document 2 discloses or suggests the lubricating performance and the flying performance, that are considered to be important characteristics in the development of the current lubricating layer molecules.

The present invention was conceived in view of the above circumstances, and provides a method for evaluating a performance of a lubricating layer, capable of evaluating the performance of the lubricating layer.

### Means for Solving the Problem

In order to solve the problem described above, the present inventors conducted diligent studies. As a result, it was found that the performance of the lubricating layer can be predicted by performing a simulation that will be described in the following.

[1] A method for evaluating a performance of a lubricating layer, comprising:
step of preparing a lubricant molecular model and a protective layer model, using quantum chemical calculation;
step of performing molecular dynamics calculation of a lubricating layer initial model constructed from the lubricant molecular model and the protective layer model; and
step of calculating an index of a degree of adsorption of lubricant molecules to the protective layer from a result of the molecular dynamics calculation.

[2] A method for evaluating a performance of a lubricating layer, comprising:
step of preparing a lubricant molecular model and a protective layer model, using quantum chemical calculation;
step of performing molecular dynamics calculation of a lubricating layer initial model constructed from the lubricant molecular model and the protective layer model; and
step of calculating an index of a degree of lubrication of lubricant molecules on the protective layer from a result of the molecular dynamics calculation.

[3] The method for evaluating the performance of the lubricating layer based on two indexes, including the index of the degree of adsorption calculated in [1], and the index of the degree of lubrication calculated in [2].

[4] The method for evaluating the performance of the lubricating layer according to any one of [1] to [3], wherein
the protective layer is a protective layer formed on a surface of a magnetic recording medium, and
the lubricant molecules are lubricant molecules for the magnetic recording medium.

### Effects of the Invention

According to features of the present invention described above, it is possible to evaluate the performance of the lubricating layer, by simulating a lubricating layer model by molecular dynamics calculation, and evaluating a superiority or inferiority of an adsorptivity or a lubricity of lubricant molecules from the calculation result. Hence, it is possible to select lubricant molecules capable of forming a lubricating layer having a performance that satisfies a target at a development site, with respect to the flying performance or the lubricating performance, without having to perform an experiment.

### BRIEF DESCRIPTION OF DRAWINGS

FIG. 1 is a diagram illustrating a unit structure of a protective layer.

FIG. 2 is a diagram illustrating procedures of a coating simulation. FIG. 2(a) is a diagram of only a protective layer, FIG. 2(b) is a diagram of lubricant molecules randomly arranged on the protective layer, FIG. 2(c) is a diagram of the coating simulation in progress, and FIG. 2(d) is a diagram of a state where the coating simulation is ended and a lubricating layer initial model is completed.

FIG. 3 is a scatter diagram illustrating both Nabs and self-diffusion coefficient of a simulation result.

FIG. 4 is a scatter diagram illustrating a correlation of the simulation result and an experimental result, and is a diagram illustrating a comparison of the Nabs and a flying property test result.

FIG. 5 is a scatter diagram illustrating a correlation between the simulation result and the experimental result, and is a diagram illustrating a comparison of the self-diffusion coefficient and a lubricity test result.

### MODE OF CARRYING OUT THE INVENTION Hereinafter, embodiments of the present

invention will be described. In the following embodiments, a method for simulating a lubricating layer model by molecular dynamics calculation, and a method for determining superiority or inferiority of lubricant molecules by utilizing a result calculated by this method, will be disclosed. The present embodiment is not limited to the following description, and can be appropriately modified without departing from the subject matter of the present invention. In addition, in the present specification, “to” indicating a numerical range includes numerical values described before and after this term as a lower limit value and an upper limit value of the range, unless indicated otherwise.

According to one aspect, a method for evaluating a performance of a lubricating layer according to embodiments of the present invention includes step of constructing a lubricant molecular model and a protective layer model using quantum chemical calculation, step of performing molecular dynamics calculation of a lubricating layer initial model constructed from the lubricant molecular model and the protective layer model, and step of calculating an index of a degree of adsorption of lubricant molecules to the protective layer from a result of the molecular dynamics calculation.

An example of a target of the evaluation method according to the present embodiment includes a case where the protective layer is a protective layer formed on a surface of a magnetic recording medium, the lubricant molecules are lubricant molecules for a magnetic recording medium, and the lubricating layer is manufactured using the lubricant molecules.

In the magnetic recording medium, a magnetic recording layer is formed on a substrate, the protective layer using carbon or the like is formed on the magnetic recording layer, and the lubricating layer is formed by coating a lubricant on a surface of the protective layer.

Accordingly, in the present embodiment, a structure model of the lubricant molecule (hereinafter referred to as a lubricant molecular model) and a structure model of the protective layer (hereinafter referred to as a protective layer model) are constructed, in order to perform a simulation of the lubricating layer of the magnetic recording medium, and an arbitrary number of lubricant molecular models are spread on the protective layer model to reproduce a state where the lubricating layer is formed on the protective layer.

### Lubricant Molecular Model and Protective Layer Model Constructing Step

In the evaluation method according to the present embodiment, a model of the lubricant molecule forming the lubricating layer, and a model of the protective layer, are first constructed.

### Lubricant Molecular Model

Examples of lubricant molecules used in the magnetic recording medium include compounds having a polar group, such as a hydroxy group or the like, at a terminal of a fluorine-based polymer having a repeat structure including a perfluoroether chain (—CF₂—), for example. A molecular weight of the lubricant molecule is approximately 1,000 to approximately 10,000, for example.

The following general chemical formula (1) represents an example of the fluorine-containing ether compound calculated in the present embodiment. This molecule is a chain macromolecule.

[Chemical Formula 1]

R1—X—R2 (1)

In the chemical formula (1), X denotes a main chain portion including the repeat structure. R1 and R2 denote a first terminal portion and a second terminal portion, respectively, and have at least one polar group typified by a hydroxyl group. The structures of R1 and R2 may be identical or may be different.

Such a molecule is structurally optimized using quantum chemical calculation, to obtain a lubricant molecular model. Because this model has information on the molecular structure and the charge of each atom, this model can be suitably used in the molecular dynamics calculation.

### Protective Layer Model

The protective layer is a layer for protecting the recording layer, and is composed of carbon atoms or silicon carbide. A unit structure of graphene or diamond carbon can be used as the unit structure of the layer composed of the carbon atoms. The graphene or diamond carbon structure is preferably doped with oxygen, nitrogen, or the like. This is because the bond of the lubricant to the polar group can be strengthened by using these doped sites as the adsorption sites.

For example, the protective layer having the graphene structure has a minimum unit including six carbon atoms as the “unit structure”.

The protective layer may have a thickness of one atomic layer, or may have a thickness of a plurality of atomic layers.

Such a unit structure of the protective layer is optimized by quantum chemical calculation, and a protective layer model is obtained by repeating the optimized unit structure in a planar direction within a range of a periodic boundary cell used in the molecular dynamics calculation that will be described later.

Because this protective layer model has information on the structure and the charge of each atom, this protective layer model can be suitably used in the molecular dynamics calculation.

### Lubricating Layer Initial Model Constructing Step

In lubricating layer initial model constructing step, an initial model of the lubricating layer (lubricating layer initial model) is constructed using the lubricant molecular model and the protective layer model constructed in the preceding step.

The lubricating layer initial model for performing the simulation may be constructed so that the lubricant molecular model is uniformly arranged to a certain extent on the protective layer model. In addition, the lubricating layer initial model is preferably constructed in the periodic boundary cell having a size such that approximately 10 to approximately 1000 lubricant molecules can be arranged therein.

As a method for arranging the lubricant molecules in the lubricating layer initial model, the lubricant molecules may be arbitrarily arranged near the protective layer model so that the individual molecules do not overlap one another, or an initial arrangement, that is stabilized using the molecular dynamics calculation or the like, may be prepared.

### Step of Performing Molecular Dynamics Calculation

In step of performing the molecular dynamics calculation, the molecular dynamics calculation is performed using the lubricating layer initial model constructed in the lubricating layer initial model construction step.

For example, the molecular dynamics calculation can be performed under the following conditions. The various conditions of the molecular dynamics calculation are not limited to the following conditions.
Cell size: 96 Å×84 Å×150 Å (periodic boundary cell)
Tracking time: 6 ns (time of one step is 1 ps, total number of steps is 6,000,000 steps)
Temperature: 300 K (assuming room temperature)
Molecular force field: GAFF force field
Calculation method of Coulomb interaction: Particle-Particle Particle-Mesh Ewald method

### Step of Calculating Index of Degree of Adsorption

The lubricant molecules are adsorbed by the interaction between the polar group in the molecules and the adsorption sites of the protective layer model.

Hence, a state where one adsorption group in the lubricant molecule is sufficiently close to the surface of the protective layer model is defined as “a state where the adsorption group is adsorbed to the protective layer”.

A method for calculating the index of the degree of adsorption of the lubricating layer composed of the lubricant molecules having a plurality of adsorption groups i (hereinafter, also referred to as adsorption groups A, B, C, . . . ) will be described below.

In the molecular dynamics calculation, the shape of the lubricant molecules and the position of the atom forming the lubricant molecules, that change with lapse of time, are tracked.

A ratio of the adsorbed state of the adsorption group A in the lubricant molecule at a certain time t in the entire lubricating layer, can be described by the following mathematical formula (I).

“Adsorption ratio of adsorption group A”=“number of adsorption groups A in adsorbed state”/“total number of lubricant molecules” (I)

The calculated time average of the adsorption ratio of the adsorption group A is regarded as an adsorption probability of the adsorption group A of the lubricant molecule.

By following similar procedures, the adsorption probability is calculated for all of the adsorption groups, including the adsorption groups B, C, . . . .

A total sum of the adsorption probabilities of the adsorption groups is regarded as a net number of adsorption groups adsorbed to the protective layer, for one lubricant molecule.

This net number is used as an index Nabs of the degree of adsorption of the lubricating layer. That is, the index Nabs is represented by the following mathematical formula (II).

$$\begin{matrix}[ Mathematical Formula II ] & \\ Nabs = {\sum }_{i} Absorption probability of absorption group i & ( II )\end{matrix}$$

As will be described later in conjunction with exemplary implementations, there is a correlation between the index of the degree of adsorption of the lubricating layer and the value of a flying property measured by experiment. For this reason, it is possible to select lubricant molecules capable of forming a lubricating layer having a good flying performance, based on the index of the degree of adsorption calculated from the lubricating layer simulation. More particularly, as will be illustrated in the exemplary implementations described later, the degree of adsorption of lubricant molecules is relatively evaluated with reference to an index of the degree of adsorption calculated for a molecule whose experimental result is clear.

According to another aspect, the lubricant performance evaluation method according to the present embodiment includes step of constructing a lubricating layer initial model including a lubricant molecular model and a protective layer model using quantum chemical calculation, step of performing molecular dynamics calculation of the lubricating layer initial model, and step of calculating an index of a degree of lubrication of lubricant molecules on the protective layer from a result of the molecular dynamics calculation.

The lubricant molecular model and protective layer model construction step, the lubricating layer initial model construction step, and the step of performing the molecular dynamics calculation are performed in a manner similar to the steps included in the lubricant evaluation method according to the present embodiment described above.

### Step of Calculating Index of Degree of Lubrication

A lubricity of the lubricant molecule is obtained by calculating the self-diffusion coefficient of the lubricant molecule from the result of the molecular dynamics calculation, and the self-diffusion coefficient is used as the index of the degree of lubrication of the lubricating layer. From the molecular dynamics calculation of a

tracking time T, a self-diffusion coefficient D of the target lubricant molecule is calculated from the following mathematical formula (III). In the mathematical formula (III), to denotes an initial time, and r(t) denotes a position of a center of gravity of the molecule at the time t.

$$[ Mathematical Formula 2 ]$$

$$D = \frac{1}{6 T} 〈 {\vert r ( T + {t}_{o} ) - r ( {t}_{o} ) \vert}^{2} 〉 \qquad ( III )$$

As will be described later in conjunction with

the exemplary implementations, there is a correlation between a value of the lubricity measured by experiment and a value of the self-diffusion coefficient of the lubricant molecule calculated from the lubricating layer simulation. For this reason, the self-diffusion coefficient calculated from the lubricating layer simulation is used as the index of the degree of lubrication of the lubricating layer. More particularly, similar to the exemplary implementations that will be described later, the lubricity of the lubricating layer is relatively evaluated for the molecule whose value of the lubricity is measured by experiment, based on the calculated index of the degree of lubrication.

The method for evaluating the performance of the lubricating layer according to the present embodiment can also evaluate the performance of the lubricating layer, based on two indexes, including the index of the degree of adsorption and the index of the degree of lubrication described above. In this case, as will be illustrated in the exemplary implementations described later, the performance of the lubricating layer can be relatively evaluated, based on the degree of adsorption calculated for the molecule whose experimental result is clear and the index of the degree of lubrication.

### Exemplary Implementations

Hereinafter, the present embodiment will be specifically described with reference to the exemplary implementations. The present embodiment is not limited to the following exemplary implementations.

### Lubricant Molecules

A main chain portion represented by the following general chemical formula (2) or the following general chemical formula (3) is used as the main chain portion X in the general chemical formula (1) described above.

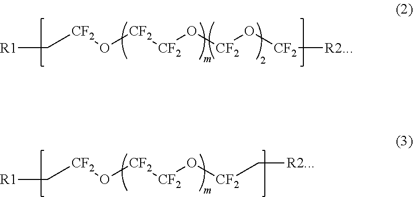

(In the general chemical formula (2) and the general chemical formula (3), m and n denote integers from 1 to 30.)

The following structures were used as the first terminal group R1 and the second terminal group R2 in the general chemical formula (1) described above.

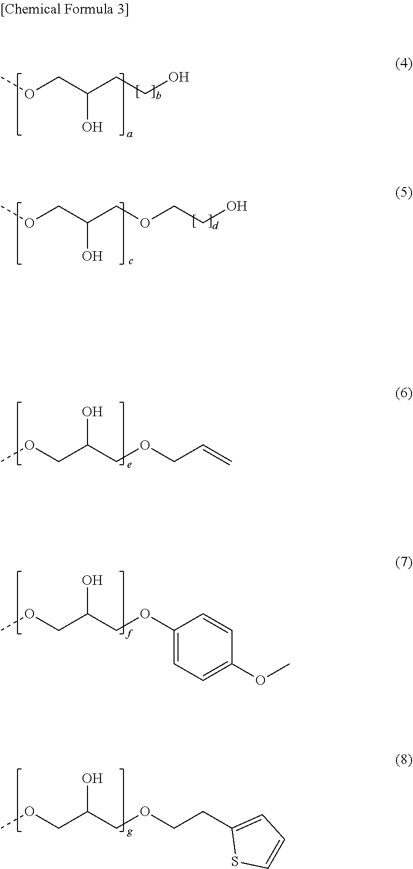

(In the general chemical formula (4), a denotes an integer from 1 to 3, and b denotes an integer from 0 to 10. In the general chemical formula (5), c denotes an integer from 1 to 3, and d denotes an integer from 0 to 10. In the general chemical formula (6), e denotes an integer from 1 to 3. In the general chemical formula (7), f denotes an integer from 1 to 3. In the general chemical formula (8), g denotes an integer from 1 to 3.)

The following simulation was performed with respect to a plurality of lubricant molecules constructed from combinations of the main chain portion X, the first terminal group R1, and the second terminal group R2 illustrated in Table 1.

### Simulation

### Protective Layer

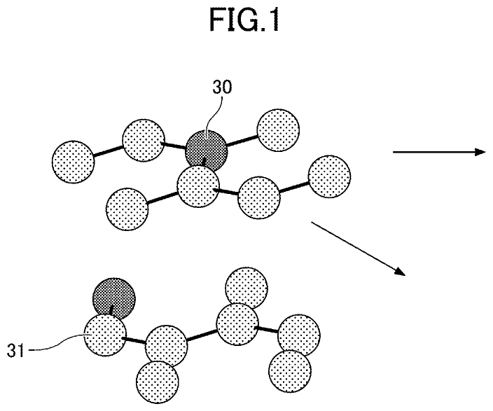

FIG. 1 is a schematic diagram for explaining the structure of the protective layer used in the simulation. As illustrated in FIG. 1, two layers of nitrogen-doped graphene were used as the protective layer in the simulation. Here, a dark-colored circle 30 indicates a nitrogen atom, a light-colored circle 31 indicates a carbon atom, and a periodic boundary condition is set such that a unit structure of each nitrogen-doped graphene is infinitely repeated in the planar direction. An arrow in FIG. 1 indicates the planar direction in which the unit structure is repeated. Each nitrogen-doped graphene is obtained by substituting a portion of the carbon atoms of the graphene with nitrogen atoms. A ratio of the nitrogen atoms in each nitrogen-doped graphene was set to 12.5% (reference value).

### Construction of Lubricant Molecular Model and Protective Layer Model

For the lubricant molecule and the unit structure of the protective layer described above, a stable structure and a charge of each atom were obtained by quantum chemical calculation. After performing optimization of the structure to calculate each of the stable structures, the atomic charge of the structure was calculated.

For the calculation of the stable structure of the lubricant molecule, B3LYP/6-31G* was used as a combination of “parameter of the density functional formalism”/“basis function”, and the atomic charge of the obtained stable structure was calculated by Minimal Basis set Mulliken population analysis. For the calculation of the stable structure of the protective layer, HSEH1PBE/6-31G* was used as the combination of “parameter of the density functional formalism”/“basis function”, and the atomic charge of the obtained stable structure was calculated by Mulliken population analysis. Gaussian 16 (registered trademark) program package of Gaussian, Inc. was used for these quantum chemical calculations.

### Step of Performing Molecular Dynamics Calculation

The step of performing the molecular dynamics calculation includes steps of performing a preliminary calculation for arranging the lubricant molecular model on the protective layer model and constructing the lubricating layer initial model (hereinafter referred to as a lubricant coating simulation), and performing a main calculation for tracking a movement of the actual lubricating layer from the constructed lubricating layer initial model (hereinafter referred to as a lubricating layer simulation).

### Lubricant Coating Simulation

The lubricating layer initial model serving as an initial structure of the lubricating layer simulation was constructed using the lubricant molecular model and the protective layer model including information on the structure and the charge of each atom constructed as described above. In order to reproduce a state where the lubricating layer covers the protective layer at room temperature (300 K), the lubricant coating simulation was performed in this exemplary implementation.

Conditions of the lubricant coating simulation were set as follows. The GAFF force field was used as the molecular force field, and a value of a depth parameter of the Lennard-Jones potential between the protective layer and the lubricating layer was scaled to ½ times (parameter of s was scaled to ½ times the original magnitude), so as to match the result of the quantum chemical calculation. In addition, the cut-off method was used for the Lennard-Jones interaction. A cut-off distance was set to 12 Å at which the interaction can be regarded as being negligible. As a temperature control method, the velocity scaling method was used to perform a simulation by an NVT ensemble in which a number of particles (N), a volume (V), and a temperature (T) are constant. The molecular dynamics calculation was performed using COGNAC engine of OCTA, that is free software.

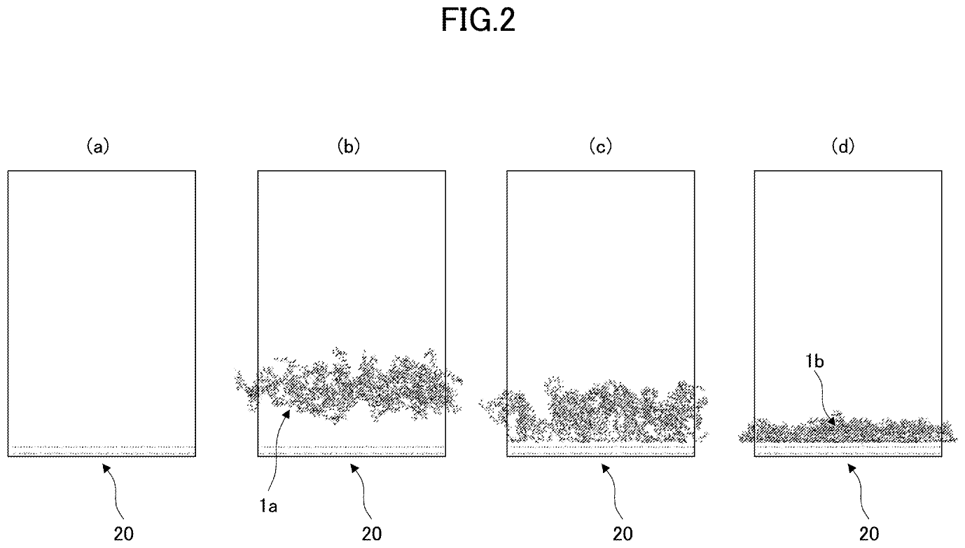

FIG. 2(a) to FIG. 2(d) are schematic diagrams for explaining the lubricant coating simulation. In FIG. 2(a) to FIG. 2(d), a reference numeral 1a denotes a lubricant molecule, a reference numeral 1b denotes a lubricating layer, and a reference numeral 20 denotes a protective layer.

First, as illustrated in FIG. 2(a), the unit structures of the protective layer stabilized by the quantum chemical calculation were spread in the periodic boundary cell of 96 Å×84 Å×150 Å, to form the protective layer 20. It was assumed that the atomic positions of the protective layer do not change during the simulation, and the atomic positions were fixed.

Next, as illustrated in FIG. 2(b), the lubricant molecules 1a were randomly arranged above the surface of the protective layer 20. In the experiment, a lubricating layer having a thin film thickness of approximately 9 ↑1 was formed, and the physical property values were measured. For comparison with the physical values, the number of molecules of the lubricant molecules 1a was adjusted so that a thickness of the lubricating layer 1b that is formed in a thermal equilibrium state at room temperature becomes approximately 9 Å.

Then, as illustrated in FIG. 2(c) and FIG. 2(d), a downward force was applied to the hydrogen atoms disposed at terminals of the lubricant molecules 1a, and the molecular dynamics calculation was performed under a room temperature condition of 300 K to lower the lubricant molecules 1a, to thereby form the lubricating layer 1b on the surface of the protective layer 20, and obtain the lubricating layer initial model. FIG. 2(c) illustrates the structure during the calculation, and FIG. 2(d) illustrates the structure of the lubricating layer 1b after the calculation of 30 ps.

### Performing Molecular Dynamics Calculation (Lubricating Layer Simulation)

In the lubricant simulation, the GAFF force field was used as the molecular force field, and a value of a depth parameter of the Lennard-Jones potential between the protective layer and the lubricating layer was scaled to ½ times (parameter of s was scaled to ½ times the original magnitude), so as to match the result of the quantum chemical calculation. In addition, the cut-off method was used for the Lennard-Jones interaction. A cut-off distance was set to 12 Å at which the interaction can be regarded as being negligible. The Particle-Particle Particle-Mesh Ewald method was used to calculate the long-range Coulomb interaction. As the temperature control method, the velocity scaling method was used to perform the simulation by the NVT ensemble in which the number of particles (N), the volume (V), and the temperature (T) are constant. The molecular dynamics calculation was performed using LAMMPS, that is free software. In the lubricating layer simulation, the

structure and behavior of the lubricating layer in the thermal equilibrium state at 300 K were reproduced, and analyzed.

In order to reach a thermal equilibrium room temperature in a short length of time, a simulation was first performed for 6 ns under a high-temperature condition of 400 K, and a simulation was then performed to gradually decrease the temperature from 400 K to 300 K by taking 1 ns. Thereafter, in order to reproduce the structure of the lubricating layer at room temperature, and to track the behavior thereof, a simulation was performed for 6 ns under the temperature condition of 300 K. In the simulation of each of the lubricant molecules illustrated in Table 1, it was confirmed that the thermal equilibrium state was sufficiently reached in approximately 3 ns.

### Calculation of Index of Each Performance

The index of the degree of adsorption and the index of the degree of lubrication of the lubricating layer were calculated.

### Calculation of Index of Degree of Adsorption

The index Nabs of the degree of adsorption of the lubricating layer was calculated by the method described above.

In this case, the distance between the protective layer and the adsorption group in a “sufficiently close state” was set to 2 Å or less for the hydroxyl group, and 3 Å or less for the amino group and the nitrile group.

### Calculation of Index of Degree of Lubrication

As illustrated in the embodiment, the self-diffusion coefficient of the lubricant molecules was calculated, and used as the index of the degree of lubrication. The displacement of lubricant molecules in the simulation for 6 ns at 300 K was used for the calculation of the self-diffusion coefficient.

Table 1 illustrates the calculated structure of the lubricating layer, and the calculation results of the index (Nabs) of the degree of adsorption and the index (self-diffusion coefficient) of the degree of lubrication. In this case, simulation No. 4 is a lubricant molecule serving as a reference of the adsorptivity. Lubricant molecules having a Nabs larger than that of No. 4 are the lubricant molecules capable of forming a lubricating layer having a more excellent adsorptivity. The simulation No. 3 is a lubricant molecule serving as a reference of the lubricity. Lubricant molecules having a self-diffusion coefficient larger than that of No. 3 are the lubricant molecules capable of forming a lubricating layer having a more excellent lubricity.

TABLE 1

| | X (general | R1 (general | R2 (general | | |
|---|---|---|---|---|---|
| | formula number) | formula number) | formula number) | | Self-diffusion |
| | Repeat numbers | Repeat numbers | Repeat numbers | Nabs | coefficient |
| Simulation No. | m, n | a~g | a~g | [no units] | [Å²/ns] |
| 1 | (2) m = 6, n = 7 | (7) f = 1 | (5) c = 1, d = 1 | 1.95 | 8.98 |
| 2 | (2) m = 6, n = 7 | (6) e = 1 | (5) c = 1, d = 1 | 1.78 | 8.85 |
| 3 | (2) m = 6, n = 7 | (8) g = 1 | (5) g = 1 | 2.63 | 10.89 |
| 4 | (2) m = 6, n = 7 | (6) e = 2 | (5) c = 1, d = 1 | 2.52 | 6.14 |
| 5 | (2) m = 6, n = 7 | (6) e = 2 | (5) c = 2, d = 1 | 2.75 | 7.59 |
| 6 | (2) m = 6, n = 7 | (7) f = 2 | (5) c = 1, d = 1 | 2.27 | 7.00 |
| 7 | (3) m = 9 | (4) a = 1, b = 0 | (5) c = 2, d = 1 | 3.17 | 11.80 |
| 8 | (2) m = 6, n = 7 | (4) a = 1, b = 0 | (4) a = 1, b = 0 | 1.70 | 12.44 |
| 9 | (3) m = 9 | (4) a = 1, b = 0 | (4) a = 1, b = 0 | 1.72 | 17.65 |
| 10 | (2) m = 6, n = 7 | (4) a = 1, b = 0 | (4) a = 2, b = 0 | 2.03 | 10.70 |
| 11 | (2) m = 6, n = 7 | (5) c = 1, d = 1 | (5) c = 1, d = 1 | 2.48 | 8.41 |
| 12 | (2) m = 6, n = 7 | (5) c = 1, d = 2 | (5) c = 1, d = 2 | 2.08 | 7.95 |
| 13 | (3) m = 9 | (5) c = 1, d = 1 | (5) c = 1, d = 1 | 2.83 | 10.62 |
| 14 | (2) m = 6, n = 7 | (5) c = 1, d = 1 | (5) c = 2, d = 1 | 3.15 | 7.47 |
| 15 | (2) m = 6, n = 7 | (5) c = 2, d = 1 | (5) c = 2, d = 1 | 3.97 | 6.54 |

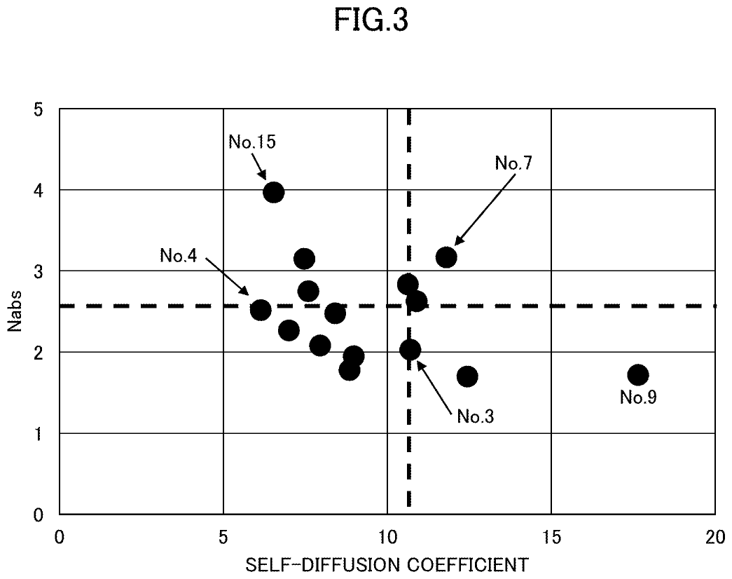

In addition, FIG. 3 is a scatter diagram illustrating both the Nabs and the self-diffusion coefficient. The ordinate indicates the Nabs, and the result of No. 4 serving as the reference is indicated by a horizontal broken line. The abscissa indicates the self-diffusion coefficient, and the result of No. 3 serving as the reference is indicated by a vertical broken line.

From the simulation results described above, it can be predicted that, for example, the lubricating layer that is formed using the lubricant of No. 15 will exhibit a performance excellent in adsorptivity, and the lubricating layer that is formed using the lubricant of No. 9 will exhibit a performance excellent in lubricity. It can be predicted that, the lubricating layer that is formed using the lubricant of No. 7 having the Nabs and the self-diffusion coefficient both exceeding the respective reference values, will exhibit performances excellent in both the adsorptivity and the lubricity.

With respect to the magnetic recording medium including the lubricating layer using the lubricant molecules described above, a flying property test and a lubricity test were performed by a conventionally known method. The results of the tests are illustrated in Table 2. The experiment No. in Table 2 and the simulation No. in Table 1 designated by the same number used the same lubricant molecules.

TABLE 2

| | Flying property | |
|---|---|---|
| Experiment No. | evaluation | Lubricity evaluation |
| 1 | 1.5 | 3 |
| 2 | 2 | 2 |
| 3 | 3 | 4 |
| 4 | 4 | 1 |
| 5 | 4 | 2 |
| 6 | 2.5 | 2.5 |
| 8 | 2 | 4 |
| 12 | 2.5 | 2.5 |

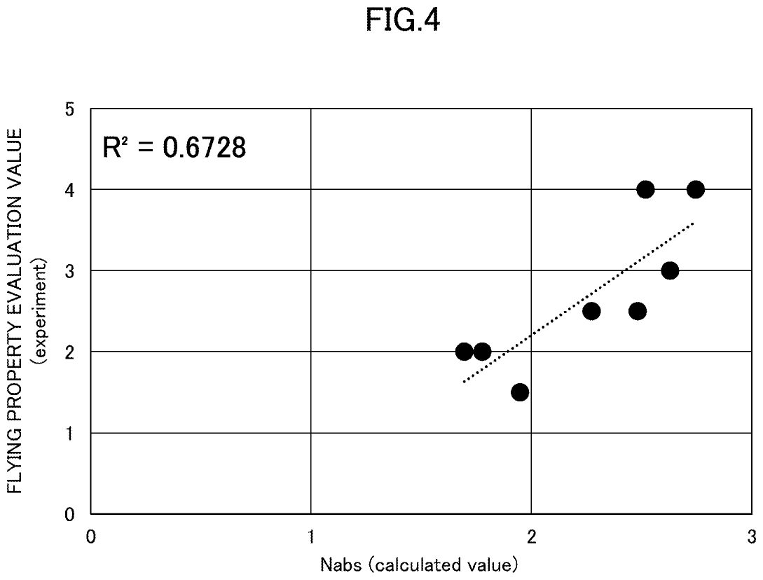

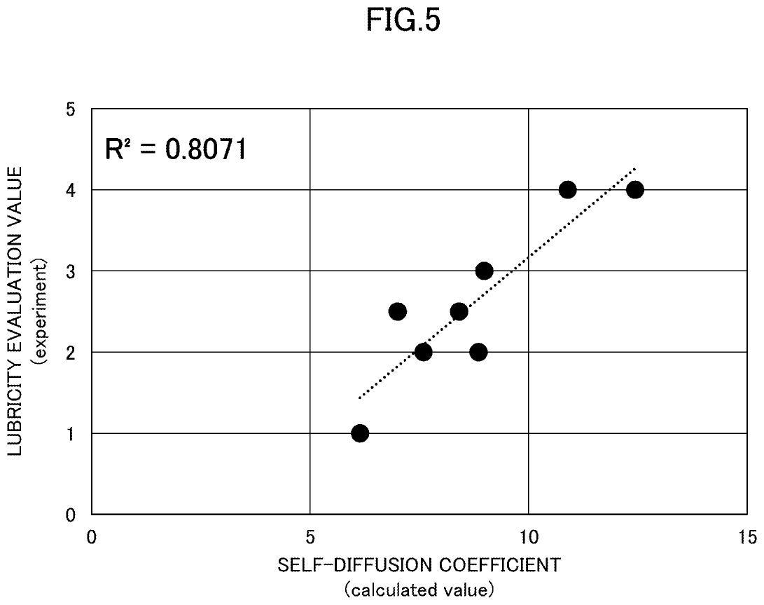

FIG. 4 and FIG. 5 illustrate scatter diagrams in which the calculation results are compared with the evaluation values of the corresponding experiments.

FIG. 4 illustrates a relationship between the evaluation value of the flying property test and the Nabs calculated by the simulation. In FIG. 4, when the linear regression formula was obtained, a coefficient of determination, R2, was approximately 0.6728, and thus, it was confirmed that there is a sufficient correlation between the flying property and the Nabs, and that the superiority or inferiority of the flying property can be determined using the Nabs.

FIG. 5 illustrates a relationship between the evaluation value of the lubricity test and the self-diffusion coefficient calculated by the simulation. In FIG. 5, when the linear regression formula was obtained, a coefficient of determination, R2, was approximately and thus, it was confirmed that there is a sufficient correlation between the lubricity and the self-diffusion coefficient, and that the superiority or inferiority of the lubricity can be determined using the self-diffusion coefficient.

Based on the above, it was confirmed that the superiority or inferiority of the flying performance and the lubricity of the lubricating layer can be predicted from the simulation result with a sufficiently high reliability.

While the embodiments of the present invention are described above, the embodiments are merely presented as examples, the present invention is not limited to the described embodiments, and various combinations, omissions, substitutions, modifications, or the like may be made without departing from the scope and the subject matter of the present invention recited in the claims. These embodiments and modifications thereof are included in the scope and subject matter of the present invention, and are included in the present invention recited in the claims and the scope of equivalents thereto.

This application is based upon and claims priority to Japanese Patent Application No. 2020-174602, filed on Oct. 16, 2020, the entire contents of which are incorporated herein by reference.

### INDUSTRIAL APPLICABILITY The method for evaluating the performance of

the lubricating layer can suitably be used as a method for evaluating a performance of a lubricating layer used in magnetic recording media or the like.

### DESCRIPTION OF REFERENCE NUMERALS

30 Nitrogen atom
31 Carbon atom
1a Lubricant molecule
1b Lubricating layer
20 Protective layer

1. A method for evaluating a performance of a lubricating layer, comprising:
preparing a lubricant molecular model and a protective layer model, using quantum chemical calculation;
performing molecular dynamics calculation of a lubricating layer initial model constructed from the lubricant molecular model and the protective layer model; and
calculating an index of a degree of adsorption of lubricant molecules to the protective layer from a result of the molecular dynamics calculation.

2. A method for evaluating a performance of a lubricating layer, comprising:
preparing a lubricant molecular model and a protective layer model, using quantum chemical calculation;
performing molecular dynamics calculation of a lubricating layer initial model constructed from the lubricant molecular model and the protective layer model; and
calculating an index of a degree of lubrication of lubricant molecules on the protective layer from a result of the molecular dynamics calculation.

3. method for evaluating the performance of the lubricating layer, comprising:
preparing a lubricant molecular model and a protective layer model, using quantum chemical calculation;
performing molecular dynamics calculation of a lubricating layer initial model constructed from the lubricant molecular model and the protective layer model;
calculating an index of a degree of adsorption of lubricant molecules to the protective layer from a result of the molecular dynamics calculation;
calculating an index of a degree of lubrication of lubricant molecules on the protective layer from a result of the molecular dynamics calculation; and
evaluating the performance of the lubricating layer based on the index of the degree of adsorption and the index of the degree of lubrication.

4. The method for evaluating the performance of the lubricating layer as claimed in claim 1, wherein
the protective layer is a protective layer formed on a surface of a magnetic recording medium, and
the lubricant molecules are lubricant molecules for the magnetic recording medium.

5. The method for evaluating the performance of the lubricating layer as claimed in claim 2, wherein
the protective layer is a protective layer formed on a surface of a magnetic recording medium, and
the lubricant molecules are lubricant molecules for the magnetic recording medium.

6. The method for evaluating the performance of the lubricating layer as claimed in claim 3, wherein
the protective layer is a protective layer formed on a surface of a magnetic recording medium, and
the lubricant molecules are lubricant molecules for the magnetic recording medium.
